(19) United States
(12) Patent Application Publication
(10) Pub. No.: US 2022/0184288 A1
(43) Pub. Date: Jun. 16, 2022

(54) OXYGENATOR FIBER MEMBRANE WITH MODIFIED SURFACE PROPERTIES

(71) Applicant: CardiacAssist, Inc., Pittsburgh, PA (US)

(72) Inventors: Michael J. Linehan, Pittsburgh, PA (US); Anthony McCoppin, Blawnox, PA (US); Patrick A. Murawski, Pittsburgh, PA (US); Robert G. Svitek, Freeport, PA (US); William Gesler, New Hudson, MI (US)

(73) Assignee: CardiacAssist, Inc., Pittsburgh, PA (US)

(21) Appl. No.: 17/686,270

(22) Filed: Mar. 3, 2022

Related U.S. Application Data
(63) Continuation of application No. PCT/US2020/052456, filed on Sep. 24, 2020.
(60) Provisional application No. 62/906,593, filed on Sep. 26, 2019.

(51) Int. Cl. A61M 1/16; B01D 69/02; B01D 69/08; B01D 63/04

(52) U.S. Cl. CPC A61M 1/1698; B01D 69/02; B01D 2325/08; B01D 63/043; B01D 2325/06; B01D 69/08

(57) ABSTRACT

A blood oxygenator includes a housing having a blood inlet, a blood outlet, a gas inlet, and a gas outlet; and a gas exchange medium having a plurality of hollow fibers in fluid communication with the gas inlet and the gas outlet. Each of the hollow fibers has a roughened outer surface configured to decrease a thickness of a boundary layer at an interface between blood and the roughened outer surface and increase a gas exchange rate at the interface.

### CROSS REFERENCE TO RELATED APPLICATIONS

This application is a continuation of International Application No. PCT/US2020/052456, filed Sep. 24, 2020, which claims the benefit of and priority to U.S. Provisional Patent Application Ser. No. 62/906,593, filed on Sep. 26, 2019, titled Oxygenator Fiber Membrane with Modified Surface Properties, the disclosure of which is incorporated herein by reference.

### BACKGROUND OF THE DISCLOSURE

### Field of the Disclosure

The present disclosure relates to blood oxygenators for use in extracorporeal membrane oxygenation (ECMO) procedures, and in particular to a fibrous membrane for use with blood oxygenators, wherein the fibrous membrane has modified surface properties to enhance gas exchange rate.

### Description of Related Art

Blood oxygenators are commonly used to accomplish the gas exchange functions normally performed by the lungs. Conventional oxygenators are commonly used in medical situations when a patient's lungs are temporarily disabled and/or incapable of performing their normal function. In some medical situations, blood oxygenators are used as a temporary gas exchange member to substitute or supplement the lung function during, for example, open heart surgery. During such procedures, vital functions of the circulatory system are assumed by an extracorporeal bypass circuit where a pump sends the patient's blood through a blood oxygenator to deliver oxygen to the patient. In other medical situations, a patient may have an indwelling catheter connected to a pump to deliver blood to a blood oxygenator. In these applications, the oxygenator can be used for an indefinite term.

Conventional blood oxygenators contain a gas exchange medium, such as a filter membrane made from hollow fibers, across which blood is flowed. The filter membrane is connected to an oxygen supply such that oxygen is diffused from the filter membrane into the blood and carbon dioxide is removed from the blood into the filter membrane.

Membrane blood oxygenators transfer oxygen into the blood as it flows over a bundle of hollow fiber membranes. The liquid side boundary layer is the limiting factor in transferring oxygen. The thickness of the boundary layer is generally dependent on the velocity of the flow, the kinematic viscosity of the fluid, and the diameter of the surface.

There is a need in the art for improved blood oxygenators having an increased gas exchange efficiency and a smaller size compared to conventional blood oxygenators.

### SUMMARY OF THE DISCLOSURE

In some examples or aspects of the present disclosure, provided is an improved blood oxygenator having an increased gas exchange efficiency and a smaller size compared to conventional blood oxygenators.

In some examples or aspects of the present disclosure, a blood oxygenator may have a housing having a blood inlet, a blood outlet, a gas inlet, and a gas outlet; and a gas exchange medium having a plurality of hollow fibers in fluid communication with the gas inlet and the gas outlet. Each of the hollow fibers may have a roughened outer surface configured to decrease a thickness of a boundary layer at an interface between blood and the roughened outer surface and increase a gas exchange rate at the interface relative to hollow fibers having a smooth outer surface.

Additionally or alternatively, in some examples the roughened outer surface of the hollow fibers includes a plurality of raised protuberances.

Additionally or alternatively, in some examples the ridges and valleys extend helically around each fiber.

Additionally or alternatively, in some examples the ridges and valleys extend circumferentially around each fiber.

Additionally or alternatively, in some examples an inner surface of each fiber is smooth.

Additionally or alternatively, in some examples the plurality of hollow fibers are arranged in multiple rows.

Additionally or alternatively, in some examples the rows of fibers are stacked on top of each other with each row angled relative to the rows in contact therewith.

Additionally or alternatively, in some examples adjacent rows are oriented perpendicular to one another.

Additionally or alternatively, in some examples the rows of hollow fibers are formed into a cylinder.

Another example of a blood oxygenator includes a housing having a blood inlet, a blood outlet disposed opposite the blood inlet, a gas inlet, and a gas outlet disposed opposite the gas outlet. The blood oxygenator also includes a gas exchange medium having a plurality of elongate hollow fibers in fluid communication with the gas inlet and the gas outlet. Each of the hollow fibers has a roughened outer surface configured to decrease a thickness of a boundary layer at an interface between blood and the roughened outer surface and increase a gas exchange rate at the interface relative to a hollow fiber with a smooth outer surface. The plurality of hollow fibers are arranged in the housing such that a direction of blood flow between the blood inlet and the blood outlet extends in a plane perpendicular to a plane of any of the hollow fibers.

Additionally or alternatively, in some examples the roughened outer surface of the hollow fibers includes a plurality of raised protuberances.

Additionally or alternatively, in some examples the roughened outer surface of the hollow fibers includes a series ridges separated by valleys.

Additionally or alternatively, in some examples the ridges and valleys extend circumferentially around each fiber.

Additionally or alternatively, in some examples the ridges and valleys extend helically around each fiber.

Additionally or alternatively, in some examples an inner surface of each fiber is smooth.

Additionally or alternatively, in some examples the plurality of hollow fibers are arranged in multiple rows.

Additionally or alternatively, in some examples the rows of fibers are stacked on top of each other with each row angled relative to the rows in contact therewith,

Additionally or alternatively, in some examples adjacent rows are oriented perpendicular to one another.

Additionally or alternatively, in some examples the rows of hollow fibers are formed into a cylinder.

Further details and advantages of the various examples or aspects described in detail herein will become clear upon reviewing the following detailed description of the various examples in conjunction with the accompanying drawing figures.

### BRIEF DESCRIPTION OF THE DRAWINGS

FIG. 1 shows a schematic representation of an extracorporeal membrane oxygenation system.

FIG. 2A shows a perspective view of an oxygenator having a fiber membrane in accordance with some examples or aspects of the present disclosure.

FIG. 2B shows an enlarged view of region 2B of the fiber membrane in FIG. 2A.

FIG. 2C shows a perspective view of an oxygenator having a fiber membrane in accordance with some examples or aspects of the present disclosure.

FIG. 3A shows a perspective view of a prior-art fiber membrane for use in an oxygenator.

FIGS. 3B and 3C show perspective views of fiber membranes for use in an oxygenator in accordance with some examples or aspects of the present disclosure.

FIG. 4A is a representative graph of boundary layer flow near a surface of a fiber of a gas exchange medium in accordance with the prior art.

FIG. 4B is a representative graph of boundary layer flow near a surface of a fiber of a gas exchange medium in accordance with some examples or aspects of the present disclosure.

### DESCRIPTION OF THE DISCLOSURE

As used herein, the singular form of “a”, “an”, and “the” include plural referents unless the context clearly dictates otherwise.

Spatial or directional terms, such as “left”, “right”, “inner”, “outer”, “above”, “below”, and the like, relate to the disclosure as shown in the drawing figures and are not to be considered as limiting as the disclosure can assume various alternative orientations.

All numbers and ranges used in the specification and claims are to be understood as being modified in all instances by the term “about”. By “about” is meant plus or minus twenty-five percent of the stated value, such as plus or minus ten percent of the stated value. However, this should not be considered as limiting to any analysis of the values under the doctrine of equivalents.

Unless otherwise indicated, all ranges or ratios disclosed herein are to be understood to encompass the beginning and ending values and any and all subranges or subratios subsumed therein. For example, a stated range or ratio of “1 to 10” should be considered to include any and all subranges or subratios between (and inclusive of) the minimum value of 1 and the maximum value of 10; that is, all subranges or subratios beginning with a minimum value of 1 or more and ending with a maximum value of 10 or less. The ranges and/or ratios disclosed herein represent the average values over the specified range and/or ratio.

The terms “first”, “second”, and the like are not intended to refer to any particular order or chronology, but refer to different conditions, properties, or elements.

The term “at least” is synonymous with “greater than or equal to”.

The term “not greater than” is synonymous with “less than or equal to”.

As used herein, “at least one of” is synonymous with “one or more of”. For example, the phrase “at least one of A, B, and C” means any one of A, B, or C, or any combination of any two or more of A, B, or C. For example, “at least one of A, B, and C” includes one or more of A alone; or one or more B alone; or one or more of C alone; or one or more of A and one or more of B; or one or more of A and one or more of C; or one or more of B and one or more of C; or one or more of all of A, B, and C.

The term “includes” is synonymous with “comprises”.

As used herein, the terms “parallel” or “substantially parallel” mean a relative angle as between two objects (if extended to theoretical intersection), such as elongated objects and including reference lines, that is from 0° to 5°, or from 0° to 3°, or from 0° to 2°, or from 0° to 1°, or from 0° to 0.5°, or from 0° to 0.25°, or from 0° to 0.1°, inclusive of the recited values.

As used herein, the terms “perpendicular” or “substantially perpendicular” mean a relative angle as between two objects at their real or theoretical intersection is from 85° to 90°, or from 87° to 90°, or from 88° to 90°, or from 89° to 90°, or from 89.5° to 90°, or from 89.75° to 90°, or from 89.9° to 90°, inclusive of the recited values.

The present disclosure is directed to a blood oxygenator suitable for use as part of an ECMO system. The oxygenator is located outside the body and provides cardiac and/or respiratory support to individuals whose heart and/or lungs are unable to function well enough on their own.

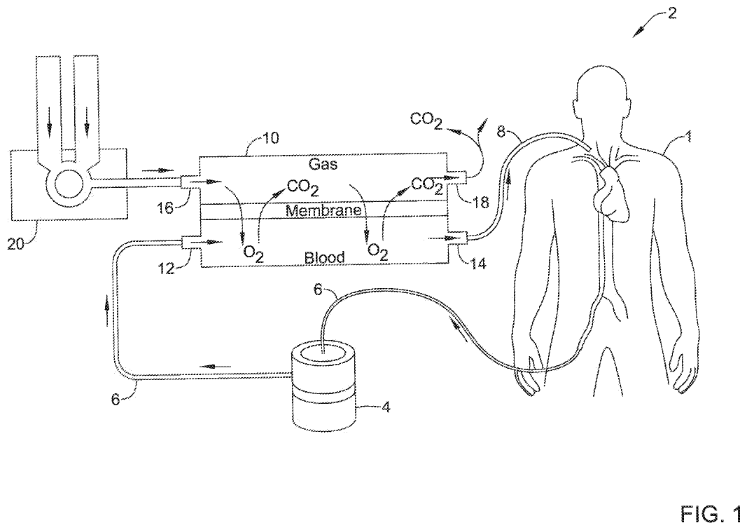

Referring now to FIG. 1, in some embodiments or aspects, an ECMO system 2 draws blood from an individual 1 at the location of a major vein or artery, typically in the groin or neck areas. Venous blood may be drawn out of the body through a cannula using, if possible, the heart, or with aide from a pump 4. The venous blood travels through a first extracorporeal tube 6 to the pump 4. The blood continues to travel through the first extracorporeal cannula 6 that is connected to the oxygenator 10. The blood flows through the oxygenator and exchanges carbon dioxide for oxygen that flows through the fibers of the oxygenator membrane before being returned to the body via a second extracorporeal cannula 8. The ECMO system 2 may be of the veno-arterial, veno-venous, arterial-arterial, or arterial-venous types. A veno-arterial system takes blood from a vein and redelivers it into an artery. A veno-venous system takes blood from a vein and redelivers it into a vein. This may occur when the heart functions properly, but the lungs cannot properly oxygenate the individual's blood. An arterial-arterial system takes blood from an artery and redelivers it into an artery. An arterial-venous system removes blood from an artery and redelivers it into a vein, and this may also occur when the heart functions properly, but the lungs do not. All of these ECMO systems typically interact with the largest veins and arteries located in the groin or neck areas, but it is contemplated that other veins or arteries of suitable size may be used.

The oxygenator may have a housing 19 with a blood inlet 12, a blood outlet 14, a gas inlet 16, and a gas outlet 18. The blood enters the oxygenator 10 by way of a blood inlet 12. The pump 4 pumps the blood through the oxygenator 10. The blood exits the oxygenator 10 by way of the blood outlet 14. The oxygenator 10 also has a gas inlet 16 connected to an oxygen source 20 that delivers oxygen through the gas inlet 16 and through the fibers of the gas exchange medium within the oxygenator 10. Because of the gas transfer that occurs within the oxygenator 10, a mix of carbon dioxide and unused oxygen is delivered out of the fiber membrane of the oxygenator 10 by way of the gas outlet 18. When travelling through the oxygenator 10, both the blood and gas travel through separate flow paths.

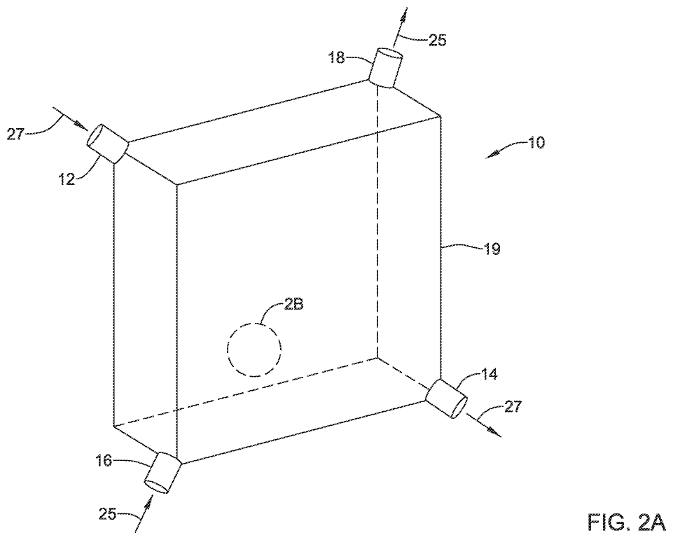

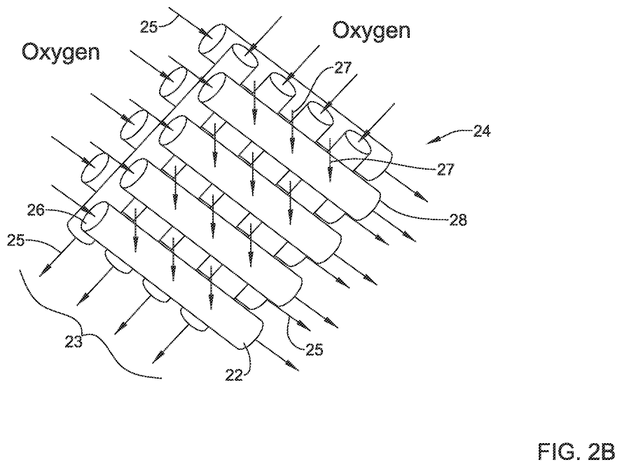

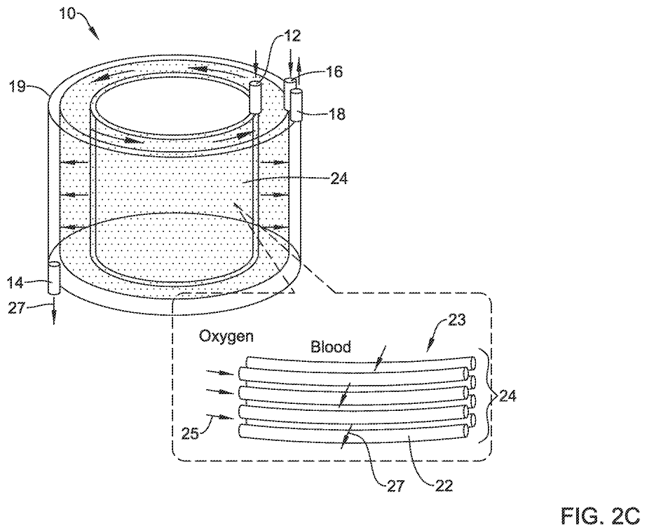

The gas flow path (indicated by the arrows 25 in FIGS. 2A-2C) is contained by numerous hollow fibers 22 that define a gas exchange medium 24 contained within the housing 19. The blood flow path (indicated with arrows 27 in FIGS. 2B and 2C) fills the space within the housing 19 between the fibers 22. The blood, by filling the spaces within the housing 19 not occupied by the fibers 22 of the gas exchange medium 24, passes over the surfaces of the fibers 22. The gas and blood flow paths may be substantially perpendicular to each other to improve oxygen transfer rate.

The fibers 22 are made of a thin, gas-permeable material that permits gas to travel into and out of the fibers 22 perpendicular to the fiber's axis. When the blood travels over the surfaces of the fibers 22, the membrane permits the gas flowing within the fibers (i.e., oxygen) to flow through the fibers 22 and into the blood. After oxygen exchange, oxygenated blood leaves the oxygenator by way of the blood outlet 14 and is delivered back into the patient's body via the second extracorporeal cannula 8.

With further reference to FIGS. 2A-2C, in some examples or aspects, the gas exchange medium 24 inside the oxygenator housing 19 may be wound into a spiral form or layered into a plurality of distinct layers. The fibers 22 of the gas exchange medium are arranged substantially parallel with each other, with each fiber 22 having a gas intake end 26 in fluid communication with a gas inlet 16 and an opposing gas outlet end 28 in fluid communication with the gas outlet 18.

In some examples or aspects of the present disclosure, the housing 19 may have a substantially rectangular shape shown in FIG. 2A. The blood inlet 12 and the blood outlet 14 are located opposite each other. The gas inlet 16 and the gas outlet 18 are also located opposite each other. Within the housing 19, multiple rows 23 of fibers 22 arranged in parallel are stacked on top of each other. Each row 23 may be angled relative to the row below it. For example, the fibers 22 of one row 23 may be substantially perpendicular to the fibers 22 of an adjacent row 23. Blood enters the oxygenator 10 by way of the blood inlet 12, and fills the space between the fibers 22 of each row of fibers 23 of the gas exchange medium 24. Oxygen flowing through the gas path of the fibers 22 diffuses through the sidewall of the fibers 22 and is absorbed by the blood flowing outside the fibers 22 before the blood exits the oxygenator 10 through the blood outlet 14.

In other examples or aspects, the housing 19 has a substantially cylindrical shape with the blood inlet 12 and the blood outlet 14 located on opposing ends of the housing 19, as shown in FIG, 2C. The gas inlet 16 and the gas outlet 18 are also located on the ends of the housing 19. One or more rows 23 of fibers 22 are formed into a mat which is then wound or bundled into a cylindrical shape.

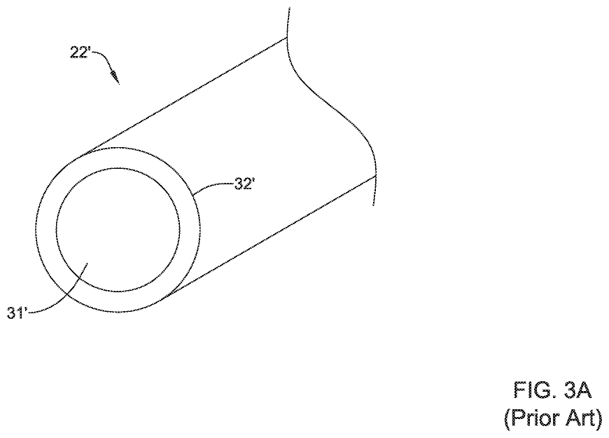

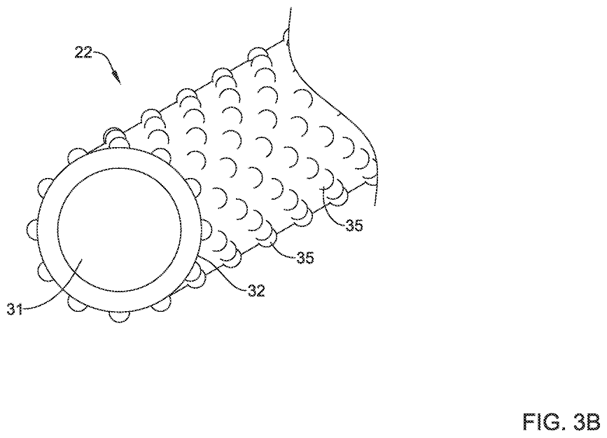

Referring to FIG. 3A, a fiber 22′ is shown in accordance with a prior art embodiment. The fiber 22′ has a substantially circular cross section with a smooth inner surface 31′ and a smooth outer surface 32′. As shown in FIG. 3B, the fiber 22 may have a roughened outer surface 32 compared to a smooth outer surface 32′ of the fiber 22′ shown in FIG. 3A. The inner surface 31 of the fiber 22 may be smooth or roughened. The roughened outer surface 32 of the fiber 22 may be defined by a plurality of raised protuberances 35 raised from a base portion of the outer surface, such as a plurality of nodules, bumps, kinks, pleats, or embossed features. The raised protuberances 35 may be arranged uniformly, non-uniformly, or randomly around the outer surface 32 and/or along the longitudinal length of the fiber 22. The raised protuberances 35 may be integral with or otherwise formed as a unitary construction with the annular wall of the fiber 22, or the raised protuberances 35 may be applied to the annular wall of the fiber 22 in a secondary process. As shown in FIG. 3C, in another example, the roughened outer surface of the fiber 122 defines a corrugated outer surface 132. The corrugated outer surface 132 may include a series of ridges 135 separated by valleys 136. In some instances, the ridges 135 and valleys 136 may extend circumferentially around the outer surface 132. In other instances, the ridges 135 and valleys 136 mat extend helically around the outer surface 132. The ridges 135 may all be the same radial height or the height of the ridges 135 may vary. The inner surface 131 may be smooth or corrugated. In some examples, the fiber 122 may be extruded to form the series of ridges 135 and valleys 136 as an integral or unitary construction with the annular wall of the fiber.

A roughened outer surface 32 increases an outer surface area of the fiber 22 and promotes mixing of the blood. In some examples, the roughened outer surface 32 may include structures, such as the above described raised protuberances 35 (e.g., nodules, bumps, kinks, pleats, embossed features) or corrugations 135, that extend 10 microns or more above a lowest point on the outer surface. In some instances, the raised protuberances 35 (e.g., nodules, bumps, kinks, pleats, embossed features) or corrugations 135 may extend 15 microns or more, 20 microns or more, or 30 microns or more above the valleys 136 and/or base of the outer surface adjacent the raised protuberances 35. For example, the distance between the valleys 136 and/or base of the outer surface and outermost extent of the ridges 135 shown in FIG. 3C may be at least 10 microns, at least 15 microns, at least 20 microns, or at least 30 microns, in some embodiments.

In some examples, the fiber 22, 122 may be a polymethylpentene (PMP) or polypropylene (PP) fiber that is formed or treated to have a roughened outer surface 32, 132. In other examples, the fiber 22, 122 may be made of one or more of polysulfone, polyethersulfone, polyarylethersulfone/polyvinylpyrrolidone, semi-synthetic membrane such as cellulose acetate or cellulose triacetate, a mixture of polyethersulfone (PES) and/or its polymer variants, combined with polyvinylpyrrolidone (PVP), polyacrilonitrile, cellulose triacetate and other cellulosics; PEPA (polyester polymer alloy); and polymethylmethacrylate (PMMA)

The function of the oxygenator 10 is to bring deoxygenated blood cells into close contact with the oxygen-filled fiber surface. The oxygen diffuses through the fiber wall and into the blood cell. The closer the cells come to the fiber surface the more efficient the gas transfer. Prior art oxygenator designs have included shakers, pulsating balloons, impellers, etc. to mix the blood and bring as many cells as close to the fibers as possible. These are often called secondary flows, as in secondary to the direction the blood was already flowing. With a roughened surface 32, 132 of the fibers 22, 122, the primary direction of blood flow will naturally create secondary flows due to the disrupted fiber surface, thereby decreasing the sluggish boundary layer and allowing more cells to get closer to the fibers and increasing the efficiency of gas transfer. The roughness of the fiber surface 32, 132 increases the friction between the outer surface of the fiber 22, 122 and the blood flowing around it. As blood flows around the fiber 22, 122, blood near the roughened outer surface 32, 132 of the fiber 22, 122 is disturbed as it moves around the fiber 22, 122.

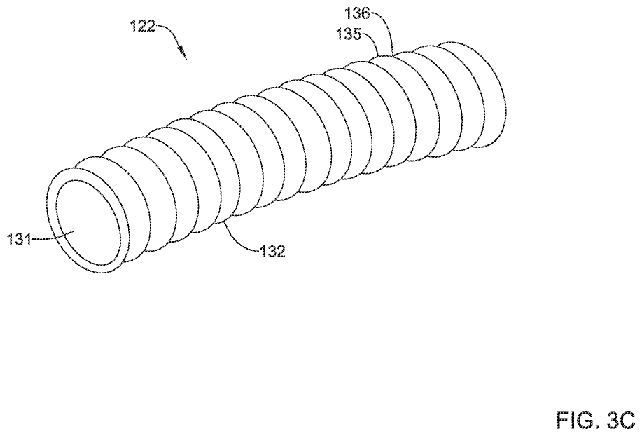

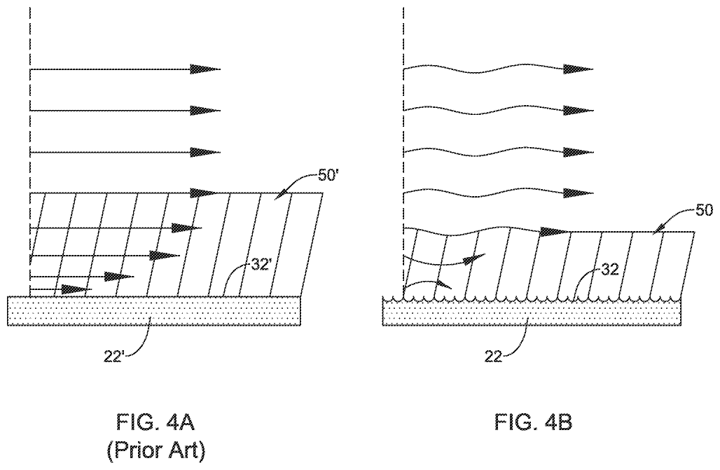

Without intending to be bound by theory, the increased friction between the blood and the roughened outer surface 32, 132 of the fiber 22, 122 creates a turbulent boundary layer 50 immediately adjacent the roughened outer surface 32 of the fiber 22, as shown in FIG. 4B. This is contrasted by a thicker boundary layer 50′ of prior art fibers 22′ due to smooth, laminar flow of blood along the smooth outer surface 32′ shown in FIG. 4A. The swirling flow of blood due to the roughened outer surface 32 of the fiber 22 decreases the thickness of the boundary layer 50 and increases the amount of oxygen that can be transferred from the fiber 22 to the blood.

While examples or aspects of an improved blood oxygenator are provided in the foregoing description, those skilled in the art may make modifications and alterations to these examples or aspects without departing from the scope and spirit of the disclosure. Accordingly, the foregoing description is intended to be illustrative rather than restrictive. The disclosure described hereinabove is defined by the appended claims, and all changes to the disclosure that fall within the meaning and the range of equivalency of the claims are to be embraced within their scope.

What is claimed is:

1. A blood oxygenator comprising:
a housing having a blood inlet, a blood outlet, a gas inlet, and a gas outlet; and
a gas exchange medium having a plurality of hollow fibers in fluid communication with the gas inlet and the gas outlet;
wherein each of the hollow fibers has a roughened outer surface configured to decrease a thickness of a boundary layer at an interface between blood and the roughened outer surface and increase a gas exchange rate at the interface relative to hollow fibers having a smooth outer surface.

2. The blood oxygenator of claim 1, wherein the roughened outer surface of the hollow fibers includes a plurality of raised protuberances.

3. The blood oxygenator of claim 1, wherein the roughened outer surface of the hollow fibers is corrugated and includes a series ridges and valleys separating the ridges.

4. The blood oxygenator of claim 3, wherein the ridges and valleys extend helically around each fiber.

5. The blood oxygenator of claim 3, wherein the ridges and valleys extend circumferentially around each fiber.

6. The blood oxygenator of claim 1, wherein an inner surface of each fiber is smooth.

7. The blood oxygenator of claim 1, wherein the plurality of hollow fibers are arranged in multiple rows.

8. The blood oxygenator of claim 7, wherein the rows of fibers are stacked on top of each other with each row angled relative to the rows in contact therewith.

9. The blood oxygenator of claim 8, wherein adjacent rows are oriented perpendicular to one another.

10. The blood oxygenator of claim 7, wherein the rows of hollow fibers are formed into a cylinder.

11. A blood oxygenator comprising:
a housing having a blood inlet, a blood outlet disposed opposite the blood inlet, a gas inlet, and a gas outlet disposed opposite the gas outlet; and
a gas exchange medium having a plurality of elongate hollow fibers in fluid communication with the gas inlet and the gas outlet, wherein each of the hollow fibers has a roughened outer surface configured to decrease a thickness of a boundary layer at an interface between blood and the roughened outer surface and increase a gas exchange rate at the interface relative to a hollow fiber with a smooth outer surface;
wherein the plurality of hollow fibers are arranged in the housing such that a direction of blood flow between the blood inlet and the blood outlet extends in a plane perpendicular to a plane of any of the hollow fibers.

12. The blood oxygenator of claim 11, wherein the roughened outer surface of the hollow fibers includes a plurality of raised protuberances.

13. The blood oxygenator of claim 11, wherein the roughened outer surface of the hollow fibers includes a series ridges separated by valleys.

14. The blood oxygenator of claim 13, wherein the ridges and valleys extend circumferentially around each fiber.

15. The blood oxygenator of claim 13, wherein the ridges and valleys extend helically around each fiber.

16. The blood oxygenator of claim 11, wherein an inner surface of each fiber is smooth.

17. The blood oxygenator of claim 11, wherein the plurality of hollow fibers are arranged in multiple rows.

18. The blood oxygenator of claim 17, wherein the rows of fibers are stacked on top of each other with each row angled relative to the rows in contact therewith,

19. The blood oxygenator of claim 17, wherein adjacent rows are oriented perpendicular to one another.

20. The blood oxygenator of claim 17, wherein the rows of hollow fibers are formed into a cylinder.
